(19) United States
(12) Patent Application Publication
(10) Pub. No.: US 2004/0098036 A1
(43) Pub. Date: May 20, 2004

(54) Device for acupuncture treatment

(76) Inventor: Turid Bergersen, Oslo (NO)

Correspondence Address: SIERRA PATENT GROUP, LTD., P O BOX 6149, STATELINE, NV, 89449, US

(21) Appl. No.: 10/472,689

(22) PCT Filed: Mar. 19, 2002
(86) PCT No.: PCT/NO02/00114

(30) Foreign Application Priority Data
Mar. 21, 2001 (NO) 2001 1427

(51) Int. Cl. A61B 17/00

(52) U.S. Cl. 606/204

(57) ABSTRACT

A device for acupressuric treatment, more specifically for stimulating the acupoints BL1 and BL2 on both sides of the root of the nose and over the eyes near the root of the nose, consists in accordance with the invention, of a resilient, bow-shaped clip that has pads arranged in pairs symmetrically about a plane of symmetry for this clip, which pads are designed to exert pressure against said acupoints when the bow is arranged over the root of the user's nose. While one set of pads faces in towards the plane of symmetry of the bow, the other set of pads faces sideways out from the plane of the bow.

### CROSS-REFERENCE TO RELATED APPLICATIONS

This application claims filing date priority benefit to Norwegian application no. 2001 1427 filed Mar. 21, 2001, and to PCT application no. PCT/NO02/00114, filed Mar. 19, 2002, under 35 U.S.C. 119 which is explicitly incorporated by reference as if set forth below.

### BACKGROUND

The present invention regards a device for acupressuric treatment, more specifically for stimulating the acupoints on both sides of the root of the nose and over the eyes.

So-called acupoints, that is acupuncture points and acupressure points, are known to exist around the eyes and on both sides of the nose, and it is also known that pressure on these points may alleviate head aches, pressure in the forehead, tiredness and irritation of the eyes, as well as other afflictions.

Glasses are known from the patent literature, which stimulate acupoints around or near the eyes in order to improve blood circulation and cell activity, thereby to alleviate afflictions such as colds, bronchitis, headaches and concentration problems. Furthermore, devices for acupressuric treatment are known from the patent literature, which devices stimulate acupoints on both sides of the nose and on the bridge of the nose in order to alleviate breathing problems (e.g., blocked nose).

From U.S. Pat. No. 4,920,956 there is known a mask-like acupressure device, the purpose of which is to alleviate afflictions such as infections of the nose. In accordance with a first embodiment, the device consists of a liquid-filled mask that may be heated and then placed on the nose. In accordance with a second embodiment, the device consists of a spectacle-like device with three electrodes that create and transmit heat to the desired acupressure points. Those points stimulated by this known device are different from those intended to be stimulated by device in accordance with the invention.

GB 2 216 800 A shows a mask-like device for massaging the muscles around the eyes in order to improve the blood circulation in these areas. The device consists of a flexible belt having a series of electrically connected elements, which elements are arranged in positions corresponding to a number of known acupuncture points around the eyes. These elements are connected to a low frequency generator, such that the elements will press against and massage the relevant acupuncture points.

GB 2 224 432 A shows a similar device to that of GB 2 216 800 A, and describes the principle of operation of the massaging elements in greater detail.

DE 4 129 229 A1 describes a device for therapeutic treatment using electroacupuncture.

CN-A 87 101 345 describes stimulation of several named acupuncture points for treatment of myopia.

### SUMMARY

The present invention regards a device of the type mentioned by way of introduction, the purpose of which is to offer a better effect than that achieved by the previously known devices. For this purpose, the invention regards a device for acupuncturic treatment, more specifically for stimulating acupoints on both sides of the root of the nose and over the eyes near the root of the nose, that is the acupoints BL1 (Jing Ming) and BL2 (Zan Zhu), in order to alleviate the beginnings of a head ache, a migraine or sinusitis. What distinguishes the device is that a resilient, bow-shaped clip has pads arranged in pairs symmetrically about a plane of symmetry for this clip, which pads are designed to exert pressure against the desired acupoints. The pads have, in an appropriate manner, been coated or impregnated with a substance that has a cooling effect on the skin, or the pads may alternatively consist essentially of a hydrophilic gel material. One set of pads faces to one side of the resilient device in a suitable manner.

### BRIEF DESCRIPTION OF THE DRAWINGS

FIG. 1 is a perspective view of an exemplary acupressure bow.

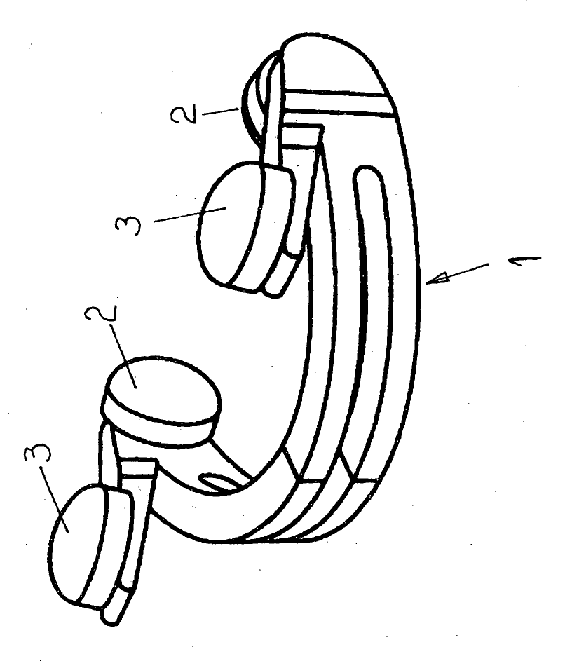

### DETAILED DESCRIPTION

The invention will be described in greater detail with reference to the appended drawing, which is a perspective view of an example of an embodiment for an acupressure bow in accordance with the invention.

The drawing shows an example of the device in accordance with the invention for acupressuric treatment, more specifically for stimulating acupoints on both sides of the root of the nose and over the eyes, that is the acupoints BL1 (Jing Ming) and BL2 (Zan Zhu), in order to relieve the beginnings of a headache, a migraine or sinusitis. The acupoints BL1 or Jing Ming are located on either side of the root of the nose, at the innermost corner of the eye, and stimulation of these points counteracts tiredness of the eyes, (e.g., “computer eyes”). The acupoints BL2 or Zan Zhu are located at the innermost edge of each eyebrow in towards the nose, and stimulation of these points is effective against sinusitis and headaches located in the forehead.

The device in accordance with the invention is in the form of a bow-shaped clip 1 having pads 2 and 3 arranged in pairs symmetrically about a plane of symmetry for the bow across the plane of the bow, which pads are designed to exert pressure against said acupoints when the bow is placed over the root of the user's nose. The pads 2 act on the acupoints BL1, while the pads 3 act on the acupoints BL2. The pads 2 face each other, in towards the plane of symmetry for the clip, while the pads 3 face sideways out from the plane of the bow.

The bow 1 is constructed from a flexible compliant or resilient material, so as to give the required pressure from the pads 2 and 3 against said acupoints.

The pads 2 and 3 are in accordance with the invention constructed wholly or substantially from a workable gel material of a type that is known per se, whereby the pads will mold themselves to the surface against which they rest, i.e. to the skin surface at the relevant acupoints, and give a good pressure effect against the acupoint. Thus the consistency of the pads is soft and workable, although the properties change somewhat according to the ambient temperatures, but still such that the pads will have these properties at most of the temperatures at which the pads will be used.

1. A device for acupressure treatment, more specifically for stimulating the acupoints BL1 and BL2 on both sides of the root of the nose and over the eyes near the root of the nose, the device comprising:
a resilient, bow-shaped clip including pads arranged in pairs symmetrically about a plane of symmetry of said clip, said pads configured to exert pressure against the accupoints responsive to said clip being arranged over the root of a user's nose.

2. The device of claim 1 wherein said pads are coated with a substance that has a cooling effect on skin.

3. The device of claim 1 wherein said pads are impregnated with a substance that has a cooling effect on skin.

4. The device of claim 1 wherein said pads comprise a hydrophilic gel material.

5. The device of claim 1 wherein one set of said pads faces sideways to one side of said resilient bow-shaped clip.
